(19) United States
(12) Patent Application Publication
(10) Pub. No.: US 2017/0071835 A1
(43) Pub. Date: Mar. 16, 2017

(54) PEG-FREE COSMETIC CLEANSING AGENTS COMPRISING BIOSURFACTANTS

(71) Applicant: Henkel AG & Co. KGaA, Duesseldorf (DE)

(72) Inventors: Heike Schelges, Willich (DE); Maria Tretyakova, Dortmund (DE); Brigitte Ludwig, Krefeld (DE)

(73) Assignee: Henkel AG & Co. KGaA, Duesseldorf (DE)

(21) Appl. No.: 15/254,512

(22) Filed: Sep. 1, 2016

(30) Foreign Application Priority Data
Sep. 14, 2015 (DE) 10 2015 217 503.0

(51) Int. Cl. A61K 8/41; A61K 8/46; A61K 8/60; A61Q 19/10; A61Q 5/02

(52) U.S. Cl. CPC A61K 8/41; A61Q 19/10; A61Q 5/02; A61K 8/60; A61K 8/602; A61K 8/463; A61K 2800/48; A61K 2800/5922; A61K 2800/596; A61K 2800/30

(57) ABSTRACT

A cosmetic cleansing agent includes biosurfactants, serving as the surfactants, in combination with an anionic surfactant and a thickener. The cosmetic cleansing agent in total includes less than 0.1 wt. %. polyethylene glycol, polypropylene glycol, ethylene oxide-containing compounds, and propylene oxide-containing compounds. A cosmetic cleansing agent having excellent foaming properties and cleansing properties may thus be provided, even though no, or substantially no, foam-promoting ethylene oxide-containing surfactants are present.

### FIELD OF THE INVENTION

The present invention generally relates to a cosmetic cleansing agent, which includes biosurfactants.

### BACKGROUND OF THE INVENTION

Aqueous cosmetic cleansing agents usually include surfactant mixtures, wherein anionic, amphoteric, non-ionic and cationic surfactants may be present. Cleansing agents including these surfactants exhibit good cleansing power, and in particular a good foaming behavior. The majority of these surfactants, however, are entirely or partially obtained from petrochemicals. Yet, sustainability in the use of cosmetic ingredients is becoming increasingly important and something that a growing number of consumers and manufacturers of cosmetic cleansing agents are demanding.

The majority of cosmetic cleansing agents available on the market include ethoxylated (=ethylene glycol-containing and/or polyethylene glycol-containing, PEG-containing) compounds, in particular as surfactants, such as sodium laureth sulfate, and optionally propoxylated (=polypropylene glycol-containing, PPG-containing) compounds. Ethoxylated compounds, such as PEG esters, are also frequently used for non-surfactant components of cosmetic cleansing agents such as thickeners. PEG itself may also be a component of cosmetic agents. However, consumers are increasingly demanding products that bear a PEG-free label since PEG-containing ingredients are linked to an influence of the barrier function of the skin. PEG-free products, however, generally have an inferior foaming behavior.

Biosurfactants are surface-active substances of microbial origin, which can be produced based on vegetable oil or sugar substrates. These substrates may in part consist of agricultural waste such as rice husks or waste waters from the sugar industry, so that in this case also no starting materials for the production of foodstuffs are lost. Biosurfactants thus meet the requirements with regard to sustainability since they are produced from renewable resources. They are used both in household cleaners, detergents and dishwashing agents (for example, U.S. Pat. No. 5,520,839, DE 19600743 A1) and in a variety of cosmetic cleansing agents (for example, WO 2014/095367 A1, WO 2013/098066 A2).

WO 2014/095367 A1 and WO 2014/118095 A1 disclose the use of biosurfactants in combination with ethylene oxide-containing anionic surfactants, such as sodium laureth sulfate.

It was an object of the present invention to provide cosmetic cleansing agents that, despite the absence of ethoxylated and propoxylated compounds, have an excellent foaming behavior and washing behavior.

Surprisingly, it was found that this can be achieved when the surfactants that the cosmetic cleansing agent includes are biosurfactants in combination with an anionic surfactant and a thickener, wherein in particular the excellent foaming behavior was not expected. At the same time, requirements with regard to sustainability and biodegradability of the ingredients included in the agent are met.

Furthermore, other desirable features and characteristics of the present invention will become apparent from the subsequent detailed description of the invention and the appended claims, taken in conjunction with this background of the invention.

### BRIEF SUMMARY OF THE INVENTION

The present invention generally relates to a cosmetic cleansing agent, which includes biosurfactants, serving as the surfactants, in combination with an anionic surfactant and a thickener, wherein the cosmetic cleansing agent in total includes less than 0.1 wt. %. polyethylene oxide (polyethylene glycol, PEG), polypropylene glycol (PPG), ethylene oxide-containing compounds, and propylene oxide-containing compounds. A cosmetic cleansing agent having excellent foaming properties and cleansing properties may thus be provided, even though no, or substantially no, foam-promoting ethylene oxide-containing surfactants are present.

Such a cosmetic cleansing agent includes, in each case based on the total weight of the cosmetic cleansing agent: 1 to 50 wt. % biosurfactant(s), 0.5 to 30 wt. % of one or more anionic surfactants, and 0.1 to 40 wt. % of one or more thickeners, wherein the cosmetic cleansing agent in total includes less than 0.1 wt. % polyethylene glycol, polypropylene glycol, ethylene oxide-containing compounds, and propylene oxide-containing compounds.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The present invention relates to:

1. A cosmetic cleansing agent that, in each case based on the total weight of the cosmetic cleansing agent, includes:

(a) 1 to 50 wt. % biosurfactant(s); and

(b) 0.5 to 30 wt. % of one or more anionic surfactants; and

(c) 0.1 to 40 wt. % of one or more thickeners,

wherein the cosmetic cleansing agent in total includes less than 0.1 wt. % polyethylene oxide (polyethylene glycol), polypropylene glycol, ethylene oxide-containing compounds, and propylene oxide-containing compounds.
2. The cosmetic cleansing agent according to item 1, including:

(a) 2 to 10 wt. % of the biosurfactant (a);

(b) 1 to 20 wt. % of the anionic surfactant (b); and

(c) 1 to 10 wt. % of the thickener (c),

each based on the total weight of the cosmetic cleansing agent.
3. The cosmetic cleansing agent according to item 1 or 2, including a glycolipid, a lipopeptide or a combination thereof as the biosurfactant (a).
4. The cosmetic cleansing agent according to any one of the preceding items, wherein the bio surfactant (a) is selected from rhamnolipids, sophorolipids, mannosylerythritols, surfactins, fatty acyl glutamates, fatty acyl glycinates, and combinations thereof
5. The cosmetic cleansing agent according to item 4, wherein the sophorolipid is a mixture of the acid form and of the lactone form, wherein 20 to 60 wt. % is present in the acid form.
6. The cleansing agent according to item 4, wherein the rhamnolipid is a mixture of mono- and di-rhamnolipids, which are each derived from 3-hydroxydodecanoic acid and/or 3-hydroxyundecanoic acid.
7. The cosmetic cleansing agent according to any one of the preceding items, wherein the anionic surfactant (b) is selected from linear alkane sulfates having 8 to 24 carbon atoms, sulfosuccinic acid monoalkyl and dialkyl esters having 8 to 24 carbon atoms in the alkyl group, acyl isethionates having 8 to 24 carbon atoms in the acyl group, acyl sarcosides having 8 to 24 carbon atoms in the acyl group, acyl glutamates having 8 to 24 carbon atoms in the acyl group, acyl glycinates having 8 to 24 carbon atoms in the acyl group, and combinations thereof
8. The cosmetic cleansing agent according to any one of the preceding items, wherein the anionic surfactant (b) is selected from acyl glutamates having 8 to 20 carbon atoms in the acyl group, alkane sulfates having 10 to 18 carbon atoms in the alkyl group, and combinations thereof
9. The cosmetic cleansing agent according to any one of the preceding items, wherein the thickener (c) is selected from xanthan gum, guar gum, gellan gum, and combinations thereof
10. The cosmetic cleansing agent according to any one of the preceding items, wherein the thickener (c) is selected from ethylene oxide-free thickeners such as Sorbitan Sesquicaprylate (INCI), Glyceryl Laurate (INCI), Glyceryl Caprylate (INCI), Glyceryl Caprate (INCI), and combinations thereof
11. The cosmetic cleansing agent according to any one of the preceding items, wherein the thickener (c) is selected from xanthan gum, Sorbitan Sesquicaprylate (INCI), and combinations thereof
12. The cosmetic cleansing agent according to any one of the preceding items, which is formulated as a shampoo or a shower gel.

The cosmetic cleansing agent according to the invention includes one or more biosurfactants as essential components.

Biosurfactants shall be understood to mean substances that are formed by microorganisms and often are also secreted from the cell. Like traditional surfactants, biosurfactants are surface-active substances that lower the surface tension of liquids and thereby promote the mixing of aqueous (hydrophilic) and water-repelling (hydrophobic) phases. Biosurfactants can be produced under mild production conditions that have a low need for energy consumption. In general, they are easily biodegradable, and the environmental compatibility thereof is very high. Moreover, they are not toxic, and no toxic by-products are generated during production of the same. Raw materials used for the microbial production of the same are carbohydrates, in particular sugar such as glucose, and/or lipophilic carbon sources such as fats, oils, partial glycerides, fatty acids, fatty alcohols, long-chain saturated or unsaturated hydrocarbons. According to the invention, the biosurfactants are preferably biosurfactants produced by way of fermentation.

The biosurfactants include glycolipids, lipopeptides, lipoproteins, fatty acids, phospholipids, neutral lipids and polymeric surfactants (such as emulsan), all of which may also be used in the present invention.

Glycolipids, which may be used in the present invention, are compounds in which one or more monosaccharide units are linked by glycosidic bonds to a lipid component. Examples of glycolipids as biosurfactants that can be used according to the invention are rhamnolipids, sophorolipids, mannosylerythritol lipids and trehalose lipids. Among these, rhamnolipids, sophorolipids, mannosylerythritol lipids and combinations thereof are preferred.

Rhamnolipids are produced by bacteria of the Pseudomonas genus, in particular Pseudomonas aeruginosa, preferably when grown on hydrophobic substrates such as n-alkanes or vegetable oils. Further glycolipids, such as glucose lipids, cellobiose lipids or trehalose lipids, are produced by different microorganisms on various substrates. Furthermore, according to the invention, mannosylerythritol lipids are preferred glycolipid biosurfactants; these are produced by bacteria of the Pseudozyma sp., Candida antarctica and Ustilago sp.

Rhamnolipids according to the present invention have the following general formula:

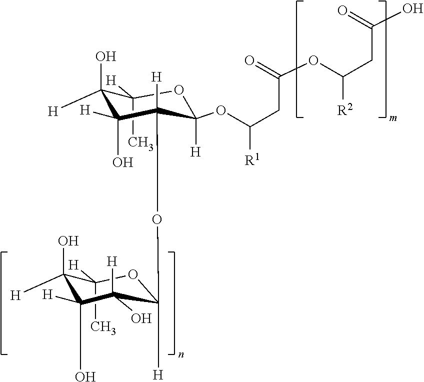

where m is 2, 1 or 0,
n is 1 or 0,
R¹ and R², independently of one another, are an identical or different organic group having 2 to 24, and preferably 5 to 13 carbon atoms, in particular a substituted or unsubstituted, branched or unbranched alkyl group, which may also be unsaturated, wherein the alkyl group is preferably a linear saturated alkyl group having 8 to 12 carbon atoms, and more preferably a nonyl or decyl group or a mixture thereof. Salts of these compounds are likewise covered by the present invention.

The term “di-rhamnolipid” in the present invention shall be understood to mean compounds of the above formula or the salts thereof, in which n is 1.

Correspondingly, “mono-rhamnolipid” in the present invention shall be understood to mean compounds of the general formula or the salts thereof, in which n is 0.

According to the invention, mixtures of mono- and di-rhamnolipids can preferably be used. The ratio of mono-rhamnolipid to di-rhamnolipid is preferably approximately 2:1 to 4:1, and more preferably 2.5:1 to 3:1. Particularly preferred are mixtures of mono- and di-rhamnolipids in which R¹ and R² independently of one another represent a linear nonyl group or a decyl group in the above formula. In the latter case, this involves rhamnolipids that are each derived from 3-hydroxydodecanoic acid and/or 3-hydroxyundecanoic acid. Such mixtures are available commercially from Agae Technologies, USA, for example, under the designation Rhamnolipid R90, R95 or R98, wherein the number in each case indicates the purity level. Rhamnolipid R90 can be used particularly preferably according to the invention.

Sophorolipids are produced by way of fermentation using yeasts such as Candida bombicola (also known as Torulopsis bombicola), Yarrowia lipolytica, Candida apicola (Torulopsis apicola), and Candida bogoriensis by growing these on sugars, hydrocarbons, vegetable oils or mixtures thereof

Sophorolipids have the below formulas (1) (lactone form) and (2) (free acid), wherein the two forms are usually present in a mixture,

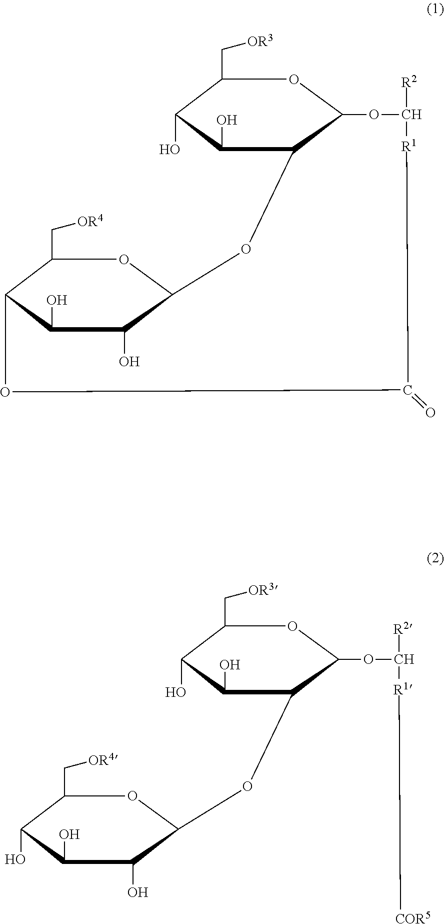

where R¹ and R^{1′}, independently of one another, represent saturated hydrocarbon chains or monounsaturated or polyunsaturated, in particular monounsaturated, hydrocarbon chains having 8 to 20, in particular 12 to 18 carbon atoms, and more preferably 14 to 18 carbon atoms, which may be linear or branched and may include one or more hydroxy groups,
R² and R^{2′}, independently of one another, represent a hydrogen atom or a saturated alkyl group or a monounsaturated or polyunsaturated, in particular monounsaturated, alkyl group having 1 to 9 carbon atoms, and more preferably 1 to 4 carbon atoms, which may be linear or branched and may include one or more hydroxy groups, and
R³, R^{3′}, R⁴ and R^{4′}, independently of one another, represent a hydrogen atom or an acetyl group.

Preferred sophorolipids are those in which R¹ and R^{1′} are a monounsaturated, linear hydrocarbon chain having 15 carbon atoms. It is furthermore preferred that R² and R^{2′} represent a methyl group or a hydrogen atom, and still more preferably each represents a methyl group.

According to the invention, preferred sophorolipids are those in which the acid form and the lactone form are present in a mixture, wherein preferably approximately 20 to approximately 60 wt. % of the sophorolipid is present in acid form and the remainder of the sophorolipid is present in lactone form.

In particular, preferred sophorolipids are those in which compounds of above formulas (1) and (2) are present in a mixture, where R¹ and R^{1′} are a monounsaturated, linear hydrocarbon chain having 14 to 18 carbon atoms, still more preferably 15 carbon atoms, R³ and R⁴ represent an acetyl group, R^{3′} and R^{4′} represent a hydrogen atom, and R² and R^{2′} represent a methyl group, and wherein approximately 20 to 60 wt. % of the sophorolipids is present in acid form.

Such sophorolipids are commercially available, for example from Soliance under the designation Sopholiance S. More precisely, the sophorolipid available from Soliance by the trade name Sopholiance S is an approximately 60 wt. % sophorolipid solution and is obtained, for example, by fermenting Candida bombicola on rapeseed oil methyl ester and glucose (INCI: Candida bombicola/glucose/methyl rapeseed ferment (and) Water). Sopholiance S is a preferred sophorolipid according to the invention.

In Soliance S, the free acid form accounts for approximately 20 wt. % in the mixture with the lactone form.

Mannosylerythritol lipids are glycolipids of the following general formula:

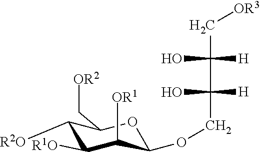

in which R¹ independently of one another represent fatty acid acyl groups having 4 to 24 carbon atoms, and preferably 8 to 12 carbon atoms, R² independently of one another represent a hydrogen atom or an acetyl group, and R³ represents a hydrogen atom or a fatty acid acyl group having 2 to 24 carbon atoms. A suitable mannosylerythritol lipid according to the invention is commercially available under the designation Ceramela-B (Toyobo) (INCI: Pseudozyma Tsukubaensis/Olive Oil/Glycerin/Soy Protein Ferment).

The biosurfactants also include the substance group of lipids and lipid derivatives, which include in particular lipopeptides. In general, lipopeptides are non-ribosomally synthesized from the respective microorganisms, for example gram-positive bacteria, in particular of the Bacillus and Streptomyces genera, gram-negative bacteria, in particular of the Pseudomonas genus, and myxobacteria, and filamentous fungi. The peptide chains are normally composed of two to forty amino acids and may be linear, cyclic or branched. In contrast to ribosomally synthesized peptide chains, the monomeric building blocks they include are often not only proteinogenic L-amino acids, but also D-amino acids and carboxylic acids and/or alpha-hydroxycarboxylic acids of all kinds. The amino acids are usually L-α- or D-α-amino acids; however, it is also possible for β-, γ- or δ-amino acids to be present, which may likewise be present in a D or else in an L configuration. The peptide chains can also include further chemical modifications; in particular, they may be glycosylated, hydrolyzed, N-methylated or N-formylated. Furthermore, thiazoline and/or oxazoline rings in various oxidation states are frequently occurring structural elements. Surfactin is a well-known lipopeptide biosurfactant, which has the following structure and is generally used as an alkali or ammonium salt:

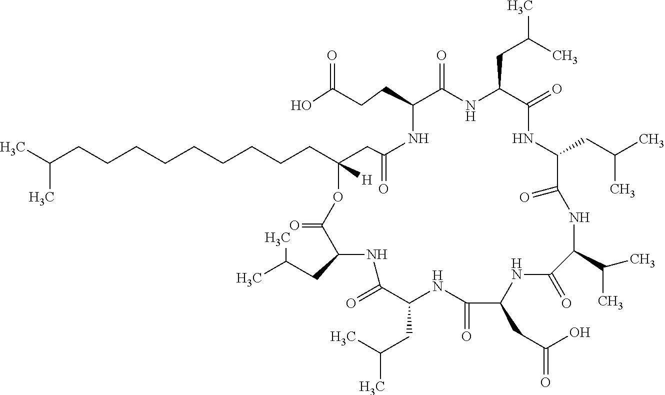 [SMILES: CC(C)CCCCCCCCC[C@@H]1CC(=O)N[C@@H](CCC(=O)O)C(=O)N[C@@H](CC(C)C)C(=O)N[C@H](CC(C)C)C(=O)N[C@@H](C(C)C)C(=O)N[C@@H](CC(=O)O)C(=O)N[C@H](CC(C)C)C(=O)N[C@@H](CC(C)C)C(=O)O1]

A surfactin that is suitable according to the invention is commercially available from Kaneka.

The lipopeptides that may preferably be used as biosurfactants according to the invention furthermore include those known as fatty acyl glutamates. These have the following general formula:

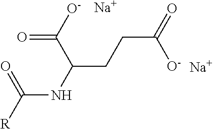

where R is a straight or branched alkyl chain having 5 to 21 carbon atoms, preferably 7 to 17 carbon atoms, and more preferably 12 to 16 or 13 to 15 carbon atoms. Fatty acyl glutamates, used as biosurfactants, are usually present in a mixture in which R has different chain lengths. The group R may also be hydroxylated, in particular monohydroxylated, wherein in this case a hydroxylation at the β-position is preferred. Fatty acyl glutamates, used as biosurfactants, are available from Modular Genetics, Inc., USA, for example.

The lipopeptides that may preferably be used as biosurfactants according to the invention furthermore include those known as fatty acyl glycinates. These have the following general formula:

RC(O)NHCH₂CO₂X,

wherein
R is a straight or branched alkyl chain having 5 to 21 carbon atoms, preferably 7 to 17 carbon atoms, and more preferably 12 to 16 or 13 to 15 carbon atoms, and
X is a cation, preferably an alkali metal or ammonium cation, more preferably a sodium or ammonium cation, or —H.

Fatty acyl glycinates, used as biosurfactants, can also be present in a mixture in which R can have different chain lengths.

Fatty acyl glycinates, used as biosurfactants, are available from Modular Genetics, Inc., USA, for example.

According to the invention, preferred cosmetic cleansing agents are those that include the following biosurfactants: rhamnolipid(s), sophorolipid(s), surfactin.

The cosmetic cleansing agent includes the biosurfactants in an amount of approximately 1 to 50 wt. %, preferably approximately 1 to 25 wt. %, more preferably approximately 2 to 15 wt. %, and more preferably 3 to 10 wt. %, based on the total weight of the cleansing agent. If biosurfactant mixtures are present, the percentage information refers to the total amount of biosurfactants present.

The cosmetic composition according to the invention includes one or more anionic surfactants as a further essential component. Aside from the fact that the anionic surfactant should not include any ethylene oxide, polyethylene oxide, propylene oxide and polypropylene oxide groups, it is not subject to any particular restrictions. The anionic surfactants customary in cosmetic cleaning agents can likewise be used with this restriction. The anionic surfactant (b) is preferably selected from linear alkane sulfates having 8 to 24 carbon atoms, and preferably 10 to 18 carbon atoms, sulfosuccinic acid monoalkyl and dialkyl esters having 8 to 24 carbon atoms, and preferably 10 to 18 carbon atoms, in the alkyl group, acyl isethionates having 8 to 24 carbon atoms, and preferably 10 to 18 carbon atoms, in the acyl group, acyl sarcosides having 8 to 24 carbon atoms, and preferably 10 to 18 carbon atoms, in the acyl group, acyl glutamates having 8 to 24 carbon atoms, and preferably 8 to 20 carbon atoms, in the acyl group, acyl glycinates having 8 to 24 carbon atoms, and preferably 10 to 18 carbon atoms, in the acyl group, and combinations thereof

The anionic surfactant (b) is particularly preferably selected from acyl glutamates having 8 to 20 carbon atoms in the acyl group, alkane sulfates having 10 to 18 carbon atoms in the alkyl group, and combinations thereof. Examples of particularly suited acyl glutamates are Sodium Cocoyl Glutamate (INCI) and Sodium Lauroyl Glutamate (INCI), Sodium Lauryl Sulfate (INCI) being one example of a particularly suited alkane sulfate. Excellent foaming properties and washing properties were observed in particular with these anionic surfactants in combination with the biosurfactants.

The cosmetic cleansing agent according to the invention includes the anionic surfactant in an amount of approximately 0.5 to 30 wt. %, preferably approximately 1 to 20 wt. %, more preferably approximately 1 to 15 wt. %, and more preferably 2 to 10 wt. %, based on the total weight of the cleansing agent. If mixtures of anionic surfactants are present, the percentage information refers to the total amount of anionic surfactants present.

In preferred embodiments of the invention, the cosmetic cleansing agent does not include any further surfactant, or at least less than 0.5 wt. % further surfactant, preferably less than 0.2 wt. %, and more preferably less than 0.1 wt. % further surfactant, in addition to the biosurfactant and the anionic surfactant. A further surfactant here shall be understood to mean non-ionic, cationic and amphoteric or zwitterionic surfactants.

The cosmetic cleansing agent according to the invention includes a thickener as a further essential component.

Preferred thickeners according to the invention are thickeners of plant origin, such as polysaccharides and celluloses (cellulose itself and the derivatives thereof), alginic acid (and the corresponding physiologically safe salts thereof, the alginates), agar agar (including the polysaccharide agarose present as the main component in agar agar), starch fractions and derivatives such as amylose, amylopectin and dextrins, karaya gum, gellan gum, locust bean gum, gum arabic, dextrans, guar gum and xanthan gum, or combinations thereof. Guar gum, gellan gum and/or xanthan gum are preferred thickeners for the present invention.

Suitable cellulose derivatives include methyl celluloses, ethyl celluloses, hydroxyalkyl celluloses (such as hydroxyethyl cellulose), methyl hydroxyalkyl celluloses and carboxymethyl celluloses (INCI: Cellulose Gum) and the physiologically safe salts thereof

Furthermore, ethylene oxide-free thickeners such as Sorbitan Sesquicaprylate (INCI), Glyceryl Laurate (INCI), Glyceryl Caprylate (INCI), Glyceryl Caprate (INCI), and combinations thereof are suitable thickeners for the cosmetic cleansing agent according to the invention. Sorbitan Sesquicaprylate is commercially available under the designation Antil Soft SC (Evonik), for example.

The thickener in the cosmetic cleansing agent of the present invention is particularly preferably selected from xanthan gum, Sorbitan Sesquicaprylate (INCI), and combinations thereof

The thickener is present in an amount of 0.1 to 40 wt. %, preferably 0.5 to 20 wt. %, more preferably 1 to 10 wt. %, and likewise preferably 1 to 5 wt. %, based on the total weight of the cosmetic cleansing agent in each case. If multiple thickeners are present, the percentage information refers to the total amount of thickeners.

One key aspect of the present invention is that the cosmetic cleansing agent in total includes 0.1 wt. % or less polyethylene oxide (polyethylene glycol), polypropylene glycol, ethylene oxide-containing compounds and propylene oxide-containing compounds, preferably less than 0.05 wt. %, and more preferably none of these substances and compounds. Ethylene oxide-containing compounds shall be understood to mean compounds that, on average, include one ethylene oxide unit (EO) per molecule, and compounds that, on average, include more than one ethylene oxide unit per molecule (polyethylene oxide-containing compounds or polyethylene glycol-containing compounds).

Propylene oxide-containing compounds shall be understood to mean compounds that, on average, include one propylene oxide unit per molecule, and compounds that, on average, include more than one propylene oxide unit per molecule (polypropylene glycol-containing compounds).

Examples of such compounds or surfactants that, according to the invention, are not present or present only in an amount of 0.1 wt. % or less are ether carboxylic acids of formula R—O—(CH₂—CH₂O)ₓ—CH₂—COOH, where R is a linear alkyl group, and x is 1 to 16, for example, alkyl polyglycol ether sulfates of formula R—O(CH₂—CH₂O)ₓ—OSO₃H, where R is an alkyl group and x is 1 to 12, for example, sulfated hydroxyalkyl polyethylene and/or hydroxyalkylene propylene glycol ethers or sulfated fatty acid alkylene glycol esters.

The feature that, in total, 0.1 wt. % or less polyethylene oxide (polyethylene glycol), polypropylene glycol, ethylene oxide-containing compounds and propylene oxide-containing compounds are present, not only includes surfactants, but also other compounds that include ethylene oxide groups or propylene oxide groups, such as thickeners.

As mentioned at the outset, in particular (poly)ethylene oxide-containing surfactants are present in cosmetic cleansing agents, given the foam-forming properties thereof. It was therefore surprising that it was also possible to provide cosmetic cleansing agents having an excellent foaming behavior and washing behavior in the absence of (poly)ethylene oxide-containing compounds. Furthermore, (poly)ethylene oxide-containing compounds frequently also enhance the ability to incorporate oils (nourishing agents) and aromatic substances. However, it was shown that the combination according to the invention of components that do not include these compounds, or include these only in a small amount, also make this possible without difficulty.

According to the invention, the cosmetic cleansing agent includes water as the cosmetic carrier. Other customary carriers may be present in embodiments of the invention, but it is preferred with respect to the biodegradability and natural availability of the raw materials if the cosmetic cleansing agent according to the invention only includes water as the carrier.

According to the invention, the cosmetic cleansing agent may include further customary ingredients of cosmetic cleansing agents. Examples of such customary ingredients are aromatic substances or perfumes, preservatives, pH regulators such as citric acid, and/or nourishing agents. The present invention, however, is not limited to these additional ingredients.

Preservatives that may be present are preferably organic acids or salts thereof, such as phenoxyethanol, methylparaben, ethylparaben, sodium benzoate, sodium salicylate and combinations thereof. If a preservative is present, this is preferably present in an amount of 0.05 to 5 wt. %, preferably 0.1 to 3 wt. %, more preferably 0.2 to 2 wt. %, and likewise preferably 0.5 to 1.0 wt. %, based on the total weight of the cleansing agent in each case. If multiple preservatives are present, the percentage information refers to the total amount of preservatives.

According to the invention, in particular natural aromatic substances are preferred as aromatic substances or perfumes that may be present in the cosmetic cleansing agent. If an aromatic substance is present, this is preferably present in an amount of 0.05 to 2 wt. %, preferably 0.1 to 1.5 wt. %, more preferably 0.2 to 1 wt. %, and likewise preferably 0.5 to 1 wt. %, based on the total weight of the cleansing agent in each case. If multiple aromatic substances are present, the percentage information refers to the total amount of aromatic substances or perfumes.

The agent can include oleosomes as nourishing agents, for example, preferably natural oleosomes such as vegetable oils, plant extracts, but also monosaccharides or oligosaccharides and/or lipids. Aloe vera extracts or olive oil shall be mentioned as examples. Glycerol is another suitable nourishing agent, which also acts as a humectant. If a nourishing agent is present, this is preferably present in an amount of 0.1 to 15 wt. %, preferably 0.5 to 10 wt. %, more preferably 1 to 7.5 wt. %, based on the total weight of the cleansing agent in each case. If multiple nourishing agents are present, the percentage information refers to the total amount of nourishing agents.

According to the invention, the cosmetic cleansing agent may be present as a shampoo, shower gel, liquid hand soap, facial cleansing agent, facial gel or other known cosmetic formulation forms.

Tabular Overview

Preferred cosmetic cleansing agents according to the invention are listed hereafter. The information is provided in % by weight in each case and refers to the active substance concentration.

| | Formula | Formula | Formula | Formula |
|---|---|---|---|---|
| | 1 | 2 | 3 | 4 |
| Biosurfactant | 1 to 50 | 1 to 25 | 2 to 15 | 3 to 10 |
| Anionic surfactant | 0.5 to 30 | 1 to 20 | 1.5 to 15 | 2 to 10 |
| Thickener | 0.1 to 40 | 0.5 to 20 | 1 to 10 | 1 to 5 |
| PEG, PPG, ethylene | ≦0.1 | ≦0.1 | ≦0.1 | ≦0.1 |
| oxide-containing and | | | | |
| propylene oxide- | | | | |
| containing compounds | | | | |
| Misc | to make | to make | to make | to make |
| | 100 | 100 | 100 | 100 |
| | Formula | Formula | Formula | Formula |
| | 5a | 6a | 7a | 8a |
| Rhamnolipid | 1 to 50 | 1 to 25 | 2 to 15 | 3 to 10 |
| Acyl glutamate having 8 | 0.5 to 30 | 1 to 20 | 1.5 to 15 | 2 to 10 |
| to 24 carbon atoms in the | | | | |
| acyl group and alkane | | | | |
| sulfate having 8 to 24 | | | | |
| carbon atoms in the alkyl | | | | |
| group | | | | |
| Guar gum, gellan gum, | 0.01 to 30 | 0.05 to 20 | 0.1 to 10 | 0.5 to 5 |
| xanthan gum, sorbitan | | | | |
| sesquicaprylate, glyceryl | | | | |
| laurate, glyceryl caprylate, | | | | |
| glyceryl caprate | | | | |
| PEG, PPG, ethylene | ≦0.05 | ≦0.05 | ≦0.05 | ≦0.05 |
| oxide-containing and | | | | |
| propylene oxide- | | | | |
| containing compounds | | | | |
| Misc | to make | to make | to make | to make |
| | 100 | 100 | 100 | 100 |
| | Formula | Formula | Formula | Formula |
| | 5b | 6b | 7b | 8b |
| Rhamnolipid | 1 to 50 | 1 to 25 | 2 to 15 | 3 to 10 |
| Acyl glycinate having 8 to | 0.5 to 30 | 1 to 20 | 1.5 to 15 | 2 to 10 |
| 24 carbon atoms in the | | | | |
| acyl group and/r alkane | | | | |
| sulfate having 8 to 24 | | | | |
| carbon atoms in the alkyl | | | | |
| group | | | | |
| Guar gum, gellan gum, | 0.01 to 30 | 0.05 to 20 | 0.1 to 10 | 0.5 to 5 |
| xanthan gum, sorbitan | | | | |
| sesquicaprylate, glyceryl | | | | |
| laurate, glyceryl caprylate, | | | | |
| glyceryl caprate | | | | |
| PEG, PPG, ethylene | ≦0.05 | ≦0.05 | ≦0.05 | ≦0.05 |
| oxide-containing and | | | | |
| propylene oxide- | | | | |
| containing compounds | | | | |
| Misc | to make | to make | to make | to make |
| | 100 | 100 | 100 | 100 |
| | Formula | Formula | Formula | Formula |
| | 5c | 6c | 7c | 8c |
| Sophorolipid | 1 to 50 | 1 to 25 | 2 to 15 | 3 to 10 |
| Acyl glutamate having 8 | 0.5 to 30 | 1 to 20 | 1.5 to 15 | 2 to 10 |
| to 24 carbon atoms in the | | | | |
| acyl group and/or alkane | | | | |
| sulfate having 8 to 24 | | | | |
| carbon atoms in the alkyl | | | | |
| group | | | | |
| Guar gum, gellan gum, | 0.01 to 30 | 0.05 to 20 | 0.1 to 10 | 0.5 to 5 |
| xanthan gum, sorbitan | | | | |
| sesquicaprylate, glyceryl | | | | |
| laurate, glyceryl caprylate, | | | | |
| glyceryl caprate | | | | |
| PEG, PPG, ethylene | ≦0.05 | ≦0.05 | ≦0.05 | ≦0.05 |
| oxide-containing and | | | | |
| propylene oxide- | | | | |
| containing compounds | | | | |
| Misc | to make | to make | to make | to make |
| | 100 | 100 | 100 | 100 |
| | Formula | Formula | Formula | Formula |
| | 5d | 6d | 7d | 8d |
| Sophorolipid | 1 to 50 | 1 to 25 | 2 to 15 | 3 to 10 |
| Acyl glycinate having 8 to | 0.5 to 30 | 1 to 20 | 1.5 to 15 | 2 to 10 |
| 24 carbon atoms in the | | | | |
| acyl group and/or alkane | | | | |
| sulfate having 8 to 24 | | | | |
| carbon atoms in the alkyl | | | | |
| group | | | | |
| Guar gum, gellan gum, | 0.01 to 30 | 0.05 to 20 | 0.1 to 10 | 0.5 to 5 |
| xanthan gum, sorbitan | | | | |
| sesquicaprylate, glyceryl | | | | |
| laurate, glyceryl caprylate, | | | | |
| glyceryl caprate | | | | |
| PEG, PPG, ethylene | ≦0.05 | ≦0.05 | ≦0.05 | ≦0.05 |
| oxide-containing and | | | | |
| propylene oxide- | | | | |
| containing compounds | | | | |
| Misc | to make | to make | to make | to make |
| | 100 | 100 | 100 | 100 |
| | Formula | Formula | Formula | Formula |
| | 5e | 6e | 7e | 8e |
| Surfactin | 1 to 50 | 1 to 25 | 2 to 15 | 3 to 10 |
| Acyl glutamate having 8 | 0.5 to 30 | 1 to 20 | 1.5 to 15 | 2 to 10 |
| to 24 carbon atoms in the | | | | |
| acyl group and/or alkane | | | | |
| sulfate having 8 to 24 | | | | |
| carbon atoms in the alkyl | | | | |
| group | | | | |
| Guar gum, gellan gum, | 0.01 to 30 | 0.05 to 20 | 0.1 to 10 | 0.5 to 5 |
| xanthan gum, sorbitan | | | | |
| sesquicaprylate, glyceryl | | | | |
| laurate, glyceryl caprylate, | | | | |
| glyceryl caprate | | | | |
| PEG, PPG, ethylene | ≦0.05 | ≦0.05 | ≦0.05 | ≦0.05 |
| oxide-containing and | | | | |
| propylene oxide- | | | | |
| containing compounds | | | | |
| Misc | to make | to make | to make | to make |
| | 100 | 100 | 100 | 100 |
| | Formula | Formula | Formula | Formula |
| | 5f | 6f | 7f | 8f |
| Surfactin | 1 to 50 | 1 to 25 | 2 to 15 | 3 to 10 |
| Acyl glycinate having 8 to | 0.5 to 30 | 1 to 20 | 1.5 to 15 | 2 to 10 |
| 24 carbon atoms in the | | | | |
| acyl group and/or alkane | | | | |
| sulfate having 8 to 24 | | | | |
| carbon atoms in the alkyl | | | | |
| group | | | | |
| Guar gum, gellan gum, | 0.01 to 30 | 0.05 to 20 | 0.1 to 10 | 0.5 to 5 |
| xanthan gum, sorbitan | | | | |
| sesquicaprylate, glyceryl | | | | |
| laurate, glyceryl caprylate, | | | | |
| glyceryl caprate | | | | |
| PEG, PPG, ethylene | ≦0.05 | ≦0.05 | ≦0.05 | ≦0.05 |
| oxide-containing and | | | | |
| propylene oxide- | | | | |
| containing compounds | | | | |
| Misc | to make | to make | to make | to make |
| | 100 | 100 | 100 | 100 |
| | Formula | Formula | Formula | Formula |
| | 9a | 10a | 11a | 12a |
| Rhamnolipid | 1 to 50 | 1 to 25 | 2 to 15 | 3 to 10 |
| Acyl glutamate having 8 | 0.5 to 30 | 1 to 20 | 1.5 to 15 | 2 to 10 |
| to 20 carbon atoms in the | | | | |
| acyl group and/or alkane | | | | |
| sulfate having 10 to 18 | | | | |
| carbon atoms in the alkyl | | | | |
| group | | | | |
| Xanthan gum and/or | 0.01 to 30 | 0.05 to 20 | 0.1 to 10 | 0.5 to 5 |
| sorbitan sesquicaprylate | | | | |
| PEG, PPG, ethylene | ≦0.05 | ≦0.05 | ≦0.05 | ≦0.05 |
| oxide-containing and | | | | |
| propylene oxide- | | | | |
| containing compounds | | | | |
| Misc | to make | to make | to make | to make |
| | 100 | 100 | 100 | 100 |
| | Formula | Formula | Formula | Formula |
| | 9b | 10b | 11b | 12b |
| Sophorolipid | 1 to 50 | 1 to 25 | 2 to 15 | 3 to 10 |
| Acyl glutamate having 8 | 0.5 to 30 | 1 to 20 | 1.5 to 15 | 2 to 10 |
| to 20 carbon atoms in the | | | | |
| acyl group and/or alkane | | | | |
| sulfate having 10 to 18 | | | | |
| carbon atoms in the alkyl | | | | |
| group | | | | |
| Xanthan gum and/or | 0.01 to 30 | 0.05 to 20 | 0.1 to 10 | 0.5 to 5 |
| sorbitan sesquicaprylate | | | | |
| PEG, PPG, ethylene | ≦0.05 | ≦0.05 | ≦0.05 | ≦0.05 |
| oxide-containing and | | | | |
| propylene oxide- | | | | |
| containing compounds | | | | |
| Misc | to make | to make | to make | to make |
| | 100 | 100 | 100 | 100 |
| | Formula | Formula | Formula | Formula |
| | 9c | 10c | 11c | 12c |
| Surfactin | 1 to 50 | 1 to 25 | 2 to 15 | 3 to 10 |
| Acyl glutamate having 8 | 0.5 to 30 | 1 to 20 | 1.5 to 15 | 2 to 10 |
| to 20 carbon atoms in the | | | | |
| acyl group and/or alkane | | | | |
| sulfate having 10 to 18 | | | | |
| carbon atoms in the alkyl | | | | |
| group | | | | |
| Xanthan gum and/or | 0.01 to 30 | 0.05 to 20 | 0.1 to 10 | 0.5 to 5 |
| sorbitan sesquicaprylate | | | | |
| PEG, PPG, ethylene | ≦0.05 | ≦0.05 | ≦0.05 | ≦0.05 |
| oxide-containing and | | | | |
| propylene oxide- | | | | |
| containing compounds | | | | |
| Misc | to make | to make | to make | to make |
| | 100 | 100 | 100 | 100 |
| | Formula | Formula | Formula | Formula |
| | 13a | 14a | 15a | 16a |
| Rhamnolipid | 1 to 50 | 1 to 25 | 2 to 15 | 3 to 10 |
| Sodium cocoyl glutamate, | 0.5 to 30 | 1 to 20 | 1.5 to 15 | 2 to 10 |
| sodium lauroyl glutamate | | | | |
| and/or sodium lauryl | | | | |
| sulfate | | | | |
| Xanthan gum and/or | 0.01 to 30 | 0.05 to 20 | 0.1 to 10 | 0.5 to 5 |
| sorbitan sesquicaprylate | | | | |
| PEG, PPG, ethylene | ≦0.05, | ≦0.05, | ≦0.05, | ≦0.05, |
| oxide-containing and | preferably | preferably | preferably | preferably |
| propylene oxide- | none | none | none | none |
| containing compounds | | | | |
| Misc | to make | to make | to make | to make |
| | 100 | 100 | 100 | 100 |
| | Formula | Formula | Formula | Formula |
| | 13b | 14b | 15b | 16b |
| Sophorolipid | 1 to 50 | 1 to 25 | 2 to 15 | 3 to 10 |
| Sodium cocoyl glutamate, | 0.5 to 30 | 1 to 20 | 1.5 to 15 | 2 to 10 |
| sodium lauroyl glutamate | | | | |
| and/or sodium lauryl | | | | |
| sulfate | | | | |
| Xanthan gum and/or | 0.01 to 30 | 0.05 to 20 | 0.1 to 10 | 0.5 to 5 |
| sorbitan sesquicaprylate | | | | |
| PEG, PPG, ethylene | ≦0.05, | ≦0.05, | ≦0.05, | ≦0.05, |
| oxide-containing and | preferably | preferably | preferably | preferably |
| propylene oxide- | none | none | none | none |
| containing compounds | | | | |
| Misc | to make | to make | to make | to make |
| | 100 | 100 | 100 | 100 |
| | Formula | Formula | Formula | Formula |
| | 13c | 14c | 15c | 16c |
| Surfactin | 1 to 50 | 1 to 25 | 2 to 15 | 3 to 10 |
| Sodium cocoyl glutamate, | 0.5 to 30 | 1 to 20 | 1.5 to 15 | 2 to 10 |
| sodium lauroyl glutamate | | | | |
| and/or sodium lauryl | | | | |
| sulfate | | | | |
| Xanthan gum and/or | 0.01 to 30 | 0.05 to 20 | 0.1 to 10 | 0.5 to 5 |
| sorbitan sesquicaprylate | | | | |
| PEG, PPG, ethylene | ≦0.05, | ≦0.05, | ≦0.05, | ≦0.05, |
| oxide-containing and | preferably | preferably | preferably | preferably |
| propylene oxide- | none | none | none | none |
| containing compounds | | | | |
| Misc | to make | to make | to make | to make |
| | 100 | 100 | 100 | 100 |

“Misc” according to the invention shall essentially be understood to mean water, if necessary in combination with another cosmetic carrier; the cosmetic carrier, however, preferably only includes water. “Misc” may optionally cover further customary ingredients of cosmetic cleansing agents such as preservatives, pH regulators such as acids, and/or aromatic substances.

PEG, PPG, the ethylene oxide-containing compounds, and the propylene oxide-containing compounds are each preferably not present, or not present in any detectable quantity.

### Examples

The following cosmetic cleansing agents shown in the table were produced. The percentage information shall be understood to mean percent by weight, in each case based on the total weight of the cleansing agent.

TABLE 1

| INCI or other | Exam- | Exam- | Exam- | Exam- | Exam- |
|---|---|---|---|---|---|
| designation | ple 1 | ple 2 | ple 3 | ple 4 | ple 5 |
| Sodium acyl | 25 | — | — | — | — |
| glutamate | | | | | |
| Sodium acyl | | 25 | | | — |
| glycinate | | | | | |
| Sulfopon 101 UP | — | — | 16.67 | 16.67 | 16.67 |
| Lauryl myristyl | | | | | |
| sulfate, sodium salt | | | | | |
| Sophorolipid - | 10 | 10 | 10 | — | — |
| Sopholiance S | | | | | |
| Rhamnolipid | — | — | — | 5.55 | — |
| Surfactin | — | — | — | — | 5.55 |
| Sodium salicylate | 0.23 | 0.23 | 0.23 | 0.23 | 0.23 |
| Sodium benzoate | 0.4 | 0.4 | 0.4 | 0.4 | 0.40 |
| Perfume | 1 | 1 | 1 | 1 | 1 |
| Antil Soft SC | 2 | 2 | 2 | 2 | — |
| Sorbitan | | | | | |
| sesquioctanoate | | | | | |
| Citric acid | 2.6 | 2.6 | 2.6 | 2.6 | 2.6 |
| Xanthan gum | 0.8 | 0.8 | — | 1 | 1 |
| Water, | to make | to make | to make | to make | to make |
| demineralized | 100 | 100 | 100 | 100 | 100 |

In all the examples, an outstanding overall cosmetic performance, including foaming behavior, washing behavior and sensation on the skin/the hair, was observed.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic cleansing agent including, in each case based on the total weight of the cosmetic cleansing agent:
(a) 1 to 50 wt. % biosurfactant(s);
(b) 0.5 to 30 wt. % of one or more anionic surfactants; and
(c) 0.1 to 40 wt. % of one or more thickeners,
wherein the cosmetic cleansing agent in total includes less than 0.1 wt. % polyethylene glycol, polypropylene glycol, ethylene oxide-containing compounds, and propylene oxide-containing compounds.

2. The cosmetic cleansing agent according to claim 1, including
(a) 2 to 10 wt. % of the biosurfactant (a);
(b) 1 to 20 wt. % of the anionic surfactant (b); and
(c) 1 to 10 wt. % of the thickener (c),
each based on the total weight of the cosmetic cleansing agent.

3. The cosmetic cleansing agent according to claim 1, including a glycolipid, a lipopeptide or a combination thereof as the biosurfactant (a).

4. The cosmetic cleansing agent according to claim 1, wherein the biosurfactant (a) is selected from the group consisting of rhamnolipids, sophorolipids, mannosylerythritols, surfactins, fatty acyl glutamates, fatty acyl glycinates, and combinations thereof.

5. The cosmetic cleansing agent according to claim 4, wherein the sophorolipid is a mixture of the acid form and of the lactone form, and wherein 20 to 60 wt. % is present in the acid form.

6. The cleansing agent according to claim 4, wherein the rhamnolipid is a mixture of mono- and di-rhamnolipids, which are each derived from 3-hydroxydodecanoic acid and/or 3-hydroxyundecanoic acid.

7. The cosmetic cleansing agent according to claim 1, wherein the anionic surfactant (b) is selected from the group consisting of linear alkane sulfates having 8 to 24 carbon atoms, sulfosuccinic acid monoalkyl and dialkyl esters having 8 to 24 carbon atoms in the alkyl group, acyl isethionates having 8 to 24 carbon atoms in the acyl group, acyl sarcosides having 8 to 24 carbon atoms in the acyl group, acyl glutamates having 8 to 24 carbon atoms in the acyl group, acyl glycinates having 8 to 24 carbon atoms in the acyl group, and combinations thereof.

8. The cosmetic cleansing agent according to claim 1, wherein the thickener (c) is selected from the group consisting of xanthan gum, guar gum, gellan gum, and combinations thereof.

9. The cosmetic cleansing agent according to claim 1, wherein the thickener (c) is one or more ethylene oxide-free thickeners selected from the group consisting of sorbitan sesquicaprylate, glyceryl laurate, glyceryl caprylate, glyceryl caprate, and combinations thereof.

10. The cosmetic cleansing agent according to claim 1, which is formulated as a shampoo or a shower gel.
